(19) United States
(12) Patent Application Publication
(10) Pub. No.: US 2007/0072931 A1
(43) Pub. Date: Mar. 29, 2007

(54) Pharmaceutical composition and a method for treatment of prostate cancer

(76) Inventors: Swati Sinha, Kolkata (IN); Bikas Chandra Pal, Kolkata (IN); Samir Bhattacharya, West Bengal (IN)

Correspondence Address: BRINKS HOFER GILSON & LIONE, P.O. BOX 10395, CHICAGO, IL, 60610, US

(21) Appl. No.: 11/511,882

(22) Filed: Aug. 29, 2006

(30) Foreign Application Priority Data
Sep. 1, 2005 (IN) 2335/DEL/2005

(51) Int. Cl. A61K 31/407

(52) U.S. Cl. 514/410

(57) ABSTRACT

The present invention is directed to pharmaceutical compositions and methods for treatment of prostate cancer in a subject. The pharmaceutical composition includes a therapeutically effective amount of compound mahanine, or derivatives, or analogues, or pharmaceutically acceptable salt thereof. The present invention is further directed to a method of isolating compound mahanine from Murraya koenigii.

### RELATED APPLICATIONS

The present patent document claims the benefit of the filing date of Indian Application No. 2335DEL2005, filed on Sep. 1, 2005, which is hereby incorporated by reference.

### BACKGROUND

1. Technical Field

The present invention relates to an herbal extract obtained from the leaves or any other plant part of Murraya koenigii (M. koenigii).

Further, it relates to a method of treating prostrate cancer in a subject.

The present invention also relates to a process for the preparation of a pharmaceutical composition comprising an extract obtained from the leaves or any other plant part of Murraya koenigii. More particularly, it relates to a process for isolation of mahanine from Murraya koenigii.

It also relates to a use of the compound mahanine as well as extract obtained from Murraya koenigii in the treatment of prostate cancer.

2. Background Information

One of the prevalent forms of neoplasia afflicting men above the age of 65 years is prostate cancer.

Mortality from prostate cancer results from metastasis to bones and lymph nodes. Early detection through serum testing of Prostate Specific Antigen (PSA), improved surgical intervention, radiation therapy, androgen ablation have yielded no answer to the patients who suffer from recurrent or residual cancer (Chinni S R, Li Y, et al., “Indole 3 carbinol(I3C) induced cell growth inhibition, G1 cell cycle arrest and Apoptosis in prostate cancer cells,” Oncogene 20:2927-2936 (2001)).

Popular, current therapy of androgen ablation almost invariably leads to aberrant expression and interaction of tyrosine kinase and associated ligands. Several recent studies have shown constitutively active mitogenic and cell survival signaling in prostate cancer. The delicate link between cellular proliferation and apoptosis is challenged by such cellular aberrations. The primary mechanism, i.e., prostate carcinoma by-pass apoptosis is by the up regulation of PI3Kinase/Akt survival pathway (Li Y, Sarkar F H, “Inhibition of nuclear factor κB activation in PC-3 cells by genistin is mediated via Akt signaling pathway,” Clin. Cancer. Res 8: 2369-2377 (2002)). Akt related serine-threonine in signaling cascade that regulates cell survival are important in the pathogenesis of cancer (Chinni S R, Sarkar F H, “Akt inactivation is a key event in Inole 3 carbinol induced apoptosis in PC-3 cells,” Clin. Cancer Res 8:1228-1236 (2002)). It inactivates a range of pro apoptotic proteins like Bad, forkhead transcription factor, caspase 9 (Green D R, Reed J C, “Mitochondria and Apoptosis,” Science 281:1309-1312 (1998); Thornberry N A, Lazebnik Y, “Caspases: enemies within,” Science 281:1312-1316 (1998)) while activating Bcl-2 (Adams J M, Cory S, “The Bcl-2 protein family Arbiters of cell survival,” Science 281:1322-1326 (1998)); NFγB like anti-apoptotic proteins (Datta S R, Brunet A, Greenberg M E, “Cellular survival: A play in three Akts,” Genes and Dev 13:2905-2927 (1999)).

### SUMMARY

In one embodiment, the present invention is a pharmaceutical composition useful for the treatment of prostate cancer in a subject.

In another embodiment, the present invention provides a method for use of a compound mahanine in the treatment of prostate cancer.

In another embodiment, the present invention is a method of treating prostrate cancer in a subject.

In yet another embodiment, the present invention is a process for the preparation of said pharmaceutical composition comprising an extract obtained from the leaves or any other plant part of Murraya koenigii useful for treatment of prostate cancer wherein the said process comprises homogenizing the dried leaves, or any other plant part of Murraya koenigii with water, wherein the ratio used is 1:1, followed by freeze drying.

In further embodiment, the present invention is a process for isolating of mahanine from Murraya koenigii.

In yet further embodiment, the present invention relates to determining cell death, release of cytochrome c, activation caspase cascade, cleavage of PARP DNA repair enzyme, along with the down regulation of Bcl-xl and pAkt, with the extract and the compound mahanine obtained from Murraya koenigii.

In one embodiment, the invention is a pharmaceutical composition for a treatment of prostate cancer in a subject comprising a therapeutically effective amount of a compound mahanine, its derivative, its analogue, or pharmaceutically acceptable salt of the compound mahanine, its derivative, or its analogue. The pharmaceutical composition may further include a pharmaceutically acceptable carrier, such as proteins, carbohydrates, sugar, magnesium stearate, cellulose, calcium carbonate, and starch-gelatin paste and pharmaceutical acceptable carriers, excipient, diluent, and solvent. The compound mahanine may be obtained from an extract of Murraya koenigii. Alternatively, the compound mahanine may be synthetically made.

In another embodiment, the present invention is a method of treating prostate cancer in a subject comprising administering to the subject a pharmaceutical composition comprising a therapeutically effective amount of a compound mahanine, its derivative, its analogue, or a pharmaceutically acceptable salt of the compound mahanine, its derivative, or its analogue. The step of administering the pharmaceutical composition may include administering a water-soluble form of the pharmaceutical composition at a unit dose of at least about 0.1-5.0 mg/kg body weight. The step of administering may further include administering the pharmaceutical composition in an oral, intravenous, intramuscular, or subcutaneous formulation. Preferably, the pharmaceutical composition kills an androgen-independent cell line PC 3 and an androgen-dependent cell line LNCaP, in a dose and time dependent manner. Preferably, the pharmaceutical composition inhibits phosphorylation of Akt in a dose and time dependent manner.

In yet another embodiment, the present invention is a method of isolating compound mahanine from Murraya koenigii. The method includes a) extracting fresh leaves of Murraya koenigii with methanol to obtain a methanol extract; b) concentrating the methanol extract from step (a) to obtain a residue I; c) suspending the residue I from step (b) in water; d) extracting the residue I with ethyl acetate and n-butanol to obtain an ethyl acetate layer; e) concentrating the ethyl acetate layer from step (d) to obtain a residue II; f) subjecting the residue II from step (e) to chromatography on silica gel using petroleum ether-chloroform (100:00→00:100) as an eluent to obtain fraction 4; g) subjecting the fraction 4 from step (f) to chromatography on silica gel; and h) crystallizing the fraction 4 in petrol to obtain the compound mahanine.

### BRIEF DESCRIPTION OF THE DRAWINGS

FIG. 1 is an illustration of structure of mahanine.

FIG. 2 is a graph showing dose response of PC-3 and LNCaP cell line to mahanine. Mahanine killed androgen independent cell line PC-3 and androgen dependent cell line LNCaP cells in a dose and time dependent manner. The dose used was 1, 2, 3 μg/ml in comparison to control untreated cells (0) in both the cell lines.

FIG. 3 is a graph showing cell viability assessment with mahanine. To evaluate the specificity of mahanine, its effect on other cells in primary culture, such as hepatocytes, cardiomyocytes, and skeletal muscle was examined with 9 μg/ml. Mahanine did not kill other cells, thereby suggesting its specificity for prostate cancer cells.

FIG. 4 is a picture showing inhibition of Akt phosphorylation by mahanine. Mahanine inhibited phosphorylation of Akt in a dose and time dependent manner. Mahanine showed no effect on the steady state levels of Akt protein but its serine 473 phosphorylation was inhibited at 24 hrs and by 36 hrs its activation was totally blocked.

FIG. 5 is a picture showing inhibition of Bcl-xl expression by mahanine. Mahanine induced apoptosis of PC-3 cells is affected through Bcl-xl. It is an antiapoptotic mitochondrial membrane protein that prevents the release of cytochrome c. Bcl-xl is considerably downregulated by 36 hrs thereby inducing apoptosis.

FIG. 6(a) is a picture showing induction of caspase 9 activation by mahanine. Caspase 9 activation was determined in the same time points with the same dose (3 μg/ml) of mahanine. Cleaved caspase 9 could be detected as early as 6 hrs, which steadily increased at 12 hrs and 24 hrs, and declined at 36 hrs.

FIG. 6(b) is a picture showing induction of caspase 3 activation by mahanine. The activation of caspase 3 considerably increased at 48 hrs and peak of the activity could be detected at 60 hrs.

FIG. 7 is a picture showing PARP cleavage by mahanine. Poly ADP ribosyl Polymerase (PARP) is a DNA repair enzyme responsible for nick detection and repair. It is one of the target proteins of caspase 3. Active caspase 3 causes cleavage of PARP into an 89 kDa fragment in PC-3 cells due to mahanine. The maximum cleavage of PARP was observed at 36 hrs.

### DETAILED DESCRIPTION OF THE DRAWINGS AND THE PRESENTLY PREFERRED EMBODIMENTS

Applicants discovered, isolated, and purified mahanine; the active fraction from the leaves of plant M. koenigii (family rutaceae). Surprisingly, both leaf extracted fraction from M. koenigii and the purified compound mahanine, a carbazole alkaloid showed considerable activity in the induction of apoptosis in two androgen independent prostate cancer cell lines PC-3 and androgen dependent prostate cancer cell line LNCaP.

The leaf extract from M. koenigii and the purified molecule, mahanine induced apoptosis of androgen independent and androgen dependent prostate cancer cells in a time and dose dependent manner. Leaf extract caused significant apoptosis of all these cell lines (75% cell death in 96 hrs) with 200 μg/ml dosage.

The leaf extract from M. koenigii induced apoptosis of androgen independent and androgen dependent prostate cancer cells in a time and dose dependent manner. Effect of the extract and purified compound on other cells, i.e., neonatal skeletal muscle cell, cardiomyocyte, hepatocyte have been examined as control cells. This suggests the specificity of the extract and mahanine for inducing apoptosis in prostate cancer cells. In in vivo experiments, there was no indication of any toxicity in mahanine treated mice (Table 3a and b). Animals were examined for acute toxicity of mahanine; however there was no change in this data until 1 month. Based on this activity, the single compound mahanine was purified. It also induced the caspase cascade and down regulation of pAkt, as well as Bcl-xl expression, the most important survival signals in cancer cells.

Accordingly, the present invention provides a pharmaceutical composition useful for the treatment of prostate cancer in a subject, wherein the pharmaceutical composition comprises a therapeutically effective amount of an extract obtained from any plant parts of Murraya koenigii or therapeutically effective amount of compound mahanine or its derivatives or analogues or pharmaceutically acceptable salt thereof, which includes salts of mahanine, its derivatives or its analogs optionally along with one or more pharmaceutically acceptable carriers. The term “derivative” refers to any compound related to the mahanine compound in that it contains the same core ring structure, but may have different substituents attached to this core. A derivative may comprise one or more substituents. Derivatives are preferably formed from the native compound mahanine either directly or by modification or partial substitution. One example of a derivative is a prodrug. The term “analogue” refers to any compound that has a structure similar, but not identical, to the native mahanine compound but differs from it in respect to certain components or side chains. Analogs may be synthesized from a different evolutionary origin.

The present invention also relates to a use of a compound mahanine in the treatment of prostate cancer. Further, it also relates to a process for the preparation of extract, and therefore a molecule of mahanine, from the leaf extract or any other plant parts of Murraya koenigii, which efficiently kills androgen dependent and independent prostate cancer cells.

Specifically, in an embodiment of the present invention, the pharmaceutical composition comprises a therapeutically effective amount of an extract obtained from any plant parts of Murraya koenigii optionally along with one or more pharmaceutically acceptable carriers.

In another embodiment of the present invention, the dosage of the pharmaceutical composition is administered at a unit dose of at least 10-15 mg/kg body weight.

Further in another embodiment of the present invention, the pharmaceutical composition further comprises a therapeutically effective amount of compound mahanine, or its derivatives, or analogues, or pharmaceutically acceptable salt thereof, optionally along with one or more pharmaceutically acceptable carriers.

In still another embodiment of the present invention, the mahanine used is obtained from the extract of Murraya koenigii. Alternatively, the mahanine may be synthetically made.

In still another embodiment of the present invention, a dosage of the pharmaceutical composition is administered at a unit dose of at least 0.1-5 mg/kg body weight.

In still another embodiment of the present invention, a dosage of the pharmaceutical composition is administered preferably in a water-soluble form.

In still another embodiment of the present invention, the carriers are selected in such a manner that they do not interfere with the activity of fraction of Murraya koenigii extract.

In still another embodiment of the present invention, the carrier is selected from the group consisting of proteins, carbohydrates, sugar, talc, magnesium stearate, cellulose, calcium carbonate, and starch-gelatin paste and pharmaceutical acceptable carriers, excipient, diluent or solvent.

In still another embodiment of the present invention, the pharmaceutical composition may be administered via oral, intravenous, intramuscular or subcutaneous route.

In still another embodiment of the present invention, the form for oral route is selected from the group consisting of capsule, syrup, concentrate, powder and granules.

Further, the present invention provides a method of treating prostrate cancer in a subject, wherein the method comprises the step of administering to the subject a pharmaceutical composition comprising a therapeutically effective amount of an extract obtained from any plant parts of Murraya koenigii, or a therapeutically effective amount of compound mahanine, or its derivatives, or analogues, or pharmaceutically acceptable salt thereof, optionally along with one or more pharmaceutically acceptable carriers.

In an embodiment of the present invention, the method of this invention includes the step of administering to the subject a pharmaceutical composition comprising a therapeutically effective amount of an extract obtained from any plant parts of Murraya koenigii, and optionally along with one or more pharmaceutically acceptable carriers.

In another embodiment of the present invention, a dosage of the above pharmaceutical composition is administered at a unit dose of at least 10-15 mg/kg body weight.

Further, in an embodiment of the present invention, the method comprises the step of administering to the subject a pharmaceutical composition comprising a therapeutically effective amount of a compound mahanine, or its derivatives, or its analogues, or pharmaceutically acceptable salt thereof, and optionally along with one or more pharmaceutically acceptable carriers.

In still another embodiment of the present invention, the mahanine used is obtained from the extract of Murraya Koenigii. Alternative, the mahanine may be synthetically made.

In still another embodiment of the present invention, a dosage of the above formulation is administered at a unit dose of at least 0.1-5.0 mg/kg body weight.

In still another embodiment of the present invention, the dosage of the pharmaceutical composition is preferably administered in a water-soluble form.

In still another embodiment of the present invention, the carriers may be selected from the group consisting of proteins, carbohydrates, sugar, talc, magnesium stearate, cellulose, calcium carbonate, and starch-gelatin paste and pharmaceutical acceptable carriers, excipient, diluent or solvent.

In still another embodiment of the present invention, the pharmaceutical composition may be administered via oral, intravenous, intramuscular or subcutaneous route.

In still another embodiment of the present invention, the form for oral route is selected from the group consisting of capsule, syrup, concentrate, powder and granules.

In still another embodiment of the present invention, the androgen independent cell line PC 3 and androgen dependent cell line LNCaP is killed by the pharmaceutical composition of this invention in a dose and time dependent manner.

In still another embodiment of the present invention, the phosphorylation of Akt is inhibited by the pharmaceutical composition of this invention in a dose and time dependent manner.

In still another embodiment of the present invention, other cells such as hepatocytes, cardiomyocytes, and skeletal muscle are not killed by the pharmaceutical composition of this invention.

The present invention also provides a process for the preparation of a pharmaceutical composition comprising an extract obtained from the leaves or any other plant part of Murraya koenigii useful for treatment of prostate cancer, wherein the process comprises of homogenizing the dried leaves or any other plant part of Murraya koenigii with water wherein the ratio used is 1:1, followed by freeze drying.

In an embodiment of the present invention, the plant parts may be leaves, stems, fruits, or any other part of the Murraya koenigii.

In another embodiment of the present invention, the leaves of Murraya koenigii are collected from different areas of West Bengal, India.

In further another embodiment of the present invention, the leaves used are taken from fresh and/or sun shade dried leaves of Murraya koenigii.

In still another embodiment of the present invention, the anti-carcinogenic activity of the extract is confirmed by in vivo experiments.

In still another embodiment of the present invention, the use of the extract is for the treatment of prostate cancer.

Further, it also provides a process for the isolation of a compound mahanine from Murraya koenigii, wherein said process comprises the steps of:
a) extracting the fresh leaves of Murraya koenigii with methanol to obtain a methanol extract;
b) concentrating the methanol extract obtained from step (a) to obtain a residue I;
c) suspending the residue I obtained from step (b) in water followed by extraction with ethyl acetate and n-butanol to obtain an ethyl acetate layer;
d) concentrating the ethyl acetate layer obtained from step (c) to get a residue II;
e) chromatograph the ethyl acetate fraction obtained from step (d) on silica gel using petroleum ether-chloroform (100:00→00:100) as an eluent to give four fractions; and
f) subjecting a fraction 4 obtained from step (e) to chromatography on silica gel followed by crystallization in petrol to get a desired product.

In an embodiment of the present invention, the structure of the compound mahanine is confirmed by comparing its physical data as well as its infrared, NMR and mass spectral data with those of an authentic sample.

In another embodiment of the present invention, the anti-carcinogenic activity of the compound mahanine is confirmed by in vivo experiments.

Further, in an embodiment of the present invention, the use of the compound mahanine is for the treatment of prostate cancer.

The following examples are given by way of illustration of the present invention and should not be construed to limit the scope of present invention.

Examples 1 to 15 relate to the extract obtained from Murraya koenigii (Rutaceae) and biological activity thereof.

### EXAMPLE 1

### Source of Murraya koenigii (Rutaceae)

The leaves of Murraya koenigii (Rutaceae) were collected from different areas of West Bengal, India. A voucher specimen has been deposited at the Department of Medicinal Chemistry, Indian Institute of Chemical Biology, Kolkata, India.

### EXAMPLE 2

### Isolation of Extract of Murraya koenigii (Rutaceae)

The fresh leaves and all other plant parts of Murraya koenigii (1.2 Kg) were homogenized with water (1.5 lit) in a mixture-blender and freeze dried. Activity of the freeze-dried material was examined on prostate cancer cell lines.

### EXAMPLE 3

### Culturing of Human Prostate Cancer Cell Line, PC-3

Human prostate cancer cell line, PC-3 (PTEN-ve, androgen independent) from American Type Culture Collection, Manassas, Va., USA was used. Cells were grown in Dulbecco's Modified Eagle Medium (DMEM) supplemented with 10% Fetal Bovine Serum (FBS) and 1% antibiotic-antimycotic. Cells were cultured in 37° C. in an atmosphere of 5% CO₂.

### Primary Culture of Rat Neonatal Cardiomyocytes

The cardiomyocytes from 2-day-old neonatal rat were isolated by the method previously described by Yoshihiro Kimura (1994) with modifications. Briefly, heart was excised and minced in pre warmed (37° C.) Ads buffer (1.2M NaCl, 198 mM HEPES, 54 mM KCl, 8.3 mM MgSO₄, 55.4 mM glucose, 95 mM NaH₂PO₄), digested in type II Collagenase 0.05% and Pancreatin in three successive digestions of 15 minutes each. Supernatant was pooled and cells palleted at 2000 g, for 10 minutes. Cells were resuspended and plated in collagen coated T-25 flasks in Medium-199 enriched with 10% FBS and 1% antibiotic-antimycotic.

### Primary Culture of Rat Neonatal Hepatocyte

The hepatocytes from 2-day-old neonatal rats were isolated by method described previously by William E. Russell (1997) with modifications. Briefly, the livers were perfused through the portal vein with a calcium-free solution consisting of 150 mM NaCl, 2.8 mM KCl, 5.5 mM glucose, and 25 mM HEPES (pH 7.6) for 10 minutes, followed by mincing and digestion in DMEM containing 0.05% collagenase type IV for 30 minutes. The cells were centrifuged (2000 g, 10 minutes) and dispersed in DMEM enriched with 10% FBS, 1% antibiotic-antimycotic.

### Primary Culture of Neonatal Rat Skeletal Muscle

The skeletal muscles from 2-day-old neonatal rats were isolated by method described previously by William E. Russell (1997) with modifications. Briefly, Soleus muscle was dissected, minced and digested with 0.2% Type II Collagenase and 0.05% trypsin in PBS pH 7.4, 0.15M NaCl. The dispersed skeletal muscle cells were centrifuged (100 g, 10 minutes) washed and resuspended in PBS with 1% antibiotic-antimycotic. Cells were preincubated for 30 minutes at 37° C., 95% air/5% CO₂. The floating muscle cells were plated on collagen coated T-25 flasks in DMEM enriched with 10% FBS and 1% antibiotic-antimycotic.

### EXAMPLE 4

Viability of PC-3 after treatment as at the indicated times was determined by MTT assay—CellTiter 96® AQₒᵤₑᵤₛ One Solution Cell Proliferation Assay (Promega Corp., Madison, Wis.), as per manufacturer's protocol. Briefly, 10,000 cells were plated in triplicate in 96 well plates and incubated for 12 hrs in complete media. Compound MK-3 was added as indicated on replacing them to fresh complete media and incubated up to 96 hrs for varied time periods. 20 μl per well of CellTiter 96® AQₒᵤₑᵤₛ One Solution reagent. Plates were incubated for 1-4 hrs at 37° C. and humidified 5% CO₂ atmosphere. Absorbance was recorded at 490 nm with 96 well plate readers. Reference wavelength of 630 nm was used to reduce background contributed by non-specific absorbance due to cell debris.

### EXAMPLE 5

### Electrophoresis and Immunoblotting

PC-3 cells (1×10⁶ cells) were incubated with complete medium. Cells were detached using cell dissociation reagent from Sigma Chemical company St Louis, Mo., USA. They were collected by centrifugation at 1500 g for 10 minutes and boiled for 5-7 minutes in sodium dodecyl sulphate (SDS) buffer (pH 6.8). Aliquots containing 60 μg total cellular protein were separated by 10% SDS-PAGE and transferred to PVDF membrane (MILIPORE, Bedford, Mass., USA). Membrane was blocked with blocking buffer for 1 hr at room temperature and probed with desired primary antibody caspase 9, Bcl-xl, pAkt/1/2/3(ser 473), anti PARP, Cytochrome C (Santa-Cruz Biotechnology, Inc. USA). Anti caspase 3 that recognizes procaspase 3 (32 kDa) and the active cleaved caspase 3 (17 kDa) (BD Biosciences, Mountainview, Calif.). Akt, cleaved caspase 9 (Cell Signaling technology, Beverly, Mass.) overnight at 4° C. followed by alkaline phosphatase conjugated secondary antibody and detection.

### EXAMPLE 6

To search anti-carcinogenic activity from medicinal plants of India, Murraya koenigii leaves extract was selected. Efficient killing of androgen independent prostate cancer cell line PC-3 was observed. It shows the data of PC-3 cell line.

### Extract of M. koenigii Reduces PC-3 Cell Viability By Inducing Apoptosis

Extracts did not show any toxic effects on neonatal skeletal muscle cell, cardiomyocyte, hepatocyte with the same dose, i.e., 100 μg/ml M. koenigii total amounting to of 200 μg/ml of culture media, suggesting the specificity of the combined extracts for inducing apoptosis in prostate cancer cells.

### In Vivo Experiments Confirm In Vitro Results

Experiments for acute toxicity were performed as per the guidelines of WHO.

Experimental subject: Mice; Balb/C Male, female; 25 gms body weight (approx).

16 mice were divided into 2 groups; 8 animals received the dosage of @2 gm/kg body weight of combined extract. Equal volume of the vehicle (DMSO) was given to 8 animals, wherein the dose was administered orally once and then animals were sacrificed and haematological parameters checked 15 days after treatment.

TABLE 1
Haematological tests for crude extract:

| | RBC | WBC | Hb (gm/dl) |
|---|---|---|---|
| Control | 1029 | 92 | 10.69 |
| Treated | 1032 | 89 | 10.713 |

There was no indication of any toxicity in treated mice (Table 1).

### EXAMPLE 7

### Inhibition of Cell-Survival Pathway Bi-Herbal Extract Obtained from M. koenigii Treated PC-3 Cells

In cell survival pathway, Akt kinase plays an important role by inhibiting apoptotic processes. Akt phosphorylates downstream effector molecules such as pro-apoptotic protein bad effecting its inactivation. This does not permit its dimerisation with Bcl-xl that result in the inhibition of apoptotic process. The bi-herbal extract inhibited phosphorylation of Akt in a dose and time dependent manner. It showed no effect on the steady state levels of Akt protein but its serine 473 phosphorylation was inhibited at 24 hrs, and by 36 hrs its activation was totally blocked. The basal level of phosphorylation at serine 473 was predominant in PC-3 cell line. The inactivation of Akt phosphorylation could be attributed to the inactivation of cell survival pathways resulting subsequent induction of apoptosis in treated PC-3 cells.

Next, studies to investigate whether mahanine bi-herbal extract (obtained from M. koenigii) induced apoptosis of PC-3 cells is affected through Bcl-xl were performed. Bcl-xl is a mitochondrial membrane protein that maintains mitochondrial membrane integrity in survival signal pathway. The bi-herbal extract significantly decreased Bcl-xl expression at 36 hrs.

This indicates initiation of apoptotic pathway as down regulation of Bcl-xl permits disintegration of mitochondrial outer membrane that causes leakage of cytochrome c initiating the caspase cascade. The activation of caspase 3 was detected at 48 hrs, and peak of the activity could be detected at 60 hrs.

### EXAMPLE 8

Therapeutic evaluation of the preparation developed by combination of M. koenigii leaves extract for the treatment of prostate cancer (Conducted by registered Auyrvedic practitioner). 3 capsules per day (each containing 330 mg) were administered orally. Results are shown in Table 2 below.

TABLE 2

| | Weight of Prostate Before | Weight of Prostate After 2 |
|---|---|---|
| Patient | Treatment | Months of Treatment |
| A | 39 gm | 21 gm |
| B | 46 gm | 25 gm |
| C | 34 gm | 18 gm |
| D | 42 gm | 27 gm |
| E | 29 gm | 16 gm |
| F | 41 gm | 19 gm |

All the above patients reported easy flow of urine during both day and night after taking capsules for 2 months.

### EXAMPLE 9

### Isolation of Compound Mahanine

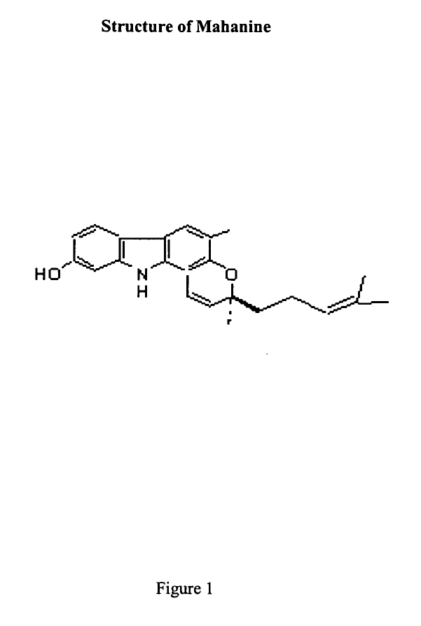

The leaves of Murraya koenigii (Rutaceae) were collected from different areas of West Bengal, India. A voucher specimen has been deposited at the Department of Medicinal Chemistry, Indian Institute of Chemical Biology, Kolkata. The fresh leaves of Murraya koenigii (3 Kg) were extracted with methanol in a mixture blender. The methanol extract was concentrated to dryness to give a residue (188 g), which was suspended in water and successively extracted with ethyl acetate and n-butanol. The ethyl acetate layer was concentrated to residue (70 g). The ethyl acetate extract was tested for bioactivity. A part of ethyl acetate fraction (16 g) was chromatographed on silica gel (250 g) using petroleum ether-chloroform (100:00→00:100) as an eluent to give four fractions, fr. 1-4, in order of elution. Fraction 4 (1.5 g) was subjected to repeated chromatography on silica gel and finally crystallization in petrol afforded a compound (0.15 g), m.p. 94-95° C., [α]_{D}+31 (chloroform) identical to the structure of mahanine (FIG. 1). Its identity was confirmed by comparing its physical data as well as its infrared (IR), ¹H NMR, ¹³C NMR and mass spectral data with those of an authentic sample (Tian-shung Wu, “Murrayamine A, B, C and (+) mahanine, carbazole alkaloid from Murraya euchrestifolia,” Phytochemistry 30,1048 (1991)). The mahanine was tested for bioactivity.

### EXAMPLE 10

The fresh leaves and all other plant parts of Murraya koenigii (1.2 Kg) were homogenized with chloroform (1.5 lit) in a mixture-blender and then sonicated in an ultrasonic bath with 3 burst each for 15 min and allow it for extraction overnight. Filtering through Whatman No. 1 filter paper separated the chloroform-extracted material. This process of extraction was repeated three times. The combined extract was evaporated to dryness in a flash evaporator under reduced pressure at 40° C. The residual substance was then dried under high vacuum. The chloroform extract (14 g) was chromatographed on silica gel column as described in Example 9. Mahanine was isolated as pure crystals (0.12 g) and was tested for bioactivity.

### EXAMPLE 11

Human prostate cancer cell line, PC-3 (PTEN-ve, androgen independent) from American Type Culture Collection, Manassas, Va., USA was used. Cells were grown in DMEM supplemented with 10% FBS and 1% antibiotic-antimycotic. Cells were cultured in 37° C. in an atmosphere of 5% CO₂.

### Primary Culture of Rat Neonatal Cardiomyocytes

The cardiomyocytes from 2-day-old neonatal rat were isolated by the method previously described by Yoshihiro Kimura (1994) with modifications. Briefly, the heart was excised and minced in pre-warmed (37° C.) Ads buffer (1.2M NaCl, 198 mM HEPES, 54 mM KCl, 8.3 mM MgSO₄, 55.4 mM glucose, 95 mM NaH₂PO₄), digested in type II Collagenase 0.05% and Pancreatin in 3-succesive digestions of 15 minutes each. Supernatant was pooled and cells palleted at 2000 g, 10 minutes. Cells were resuspended and plated in collagen coated T-25 flasks in Medium-199 enriched with 10% FBS and 1% antibiotic-antimycotic.

### Primary Culture of Rat Neonatal Hepatocytes

The hepatocytes from 2-day-old neonatal rat were isolated by method described previously by William E Russell (1997) with modifications.

Briefly, the livers were perfused through the portal vein with a calcium-free solution consisting of 150 mM NaCl, 2.8 mM KCl, 5.5 mM glucose, and 25 mM HEPES (pH 7.6) for 10 minutes, followed by mincing and digestion in DMEM containing 0.05% collagenase type IV for 30 minutes. The cells were centrifuged (2000 g, 10 minutes) and dispersed in DMEM enriched with 10% FBS, 1% antibiotic-antimycotic.

### Primary Culture of Neonatal Rat Skeletal Muscle

The skeletal muscles from 2-day-old neonatal rat were isolated by method described previously by William E. Russell (1997) with modifications.

Briefly, Soleus muscles were dissected, minced and digested with 0.2% Type II Collagenase and 0.05% trypsin in PBS, pH 7.4, 0.15M NaCl. The dispersed skeletal muscle cells were centrifuged (1000 g, 10 minutes) washed and re-suspended in PBS with 1% antibiotic-antimycotic. Cells were preincubated for 30 minutes at 37° C., 95% air/5% CO₂. The floating muscle cells were plated on collagen coated T-25 flasks in DMEM enriched with 10% FBS and 1% antibiotic-antimycotic.

### EXAMPLE 12

Viability of PC-3 after treatment as at the indicated times was determined by MTT assay—CellTiter 96® AQₒᵤₑᵤₛ One Solution Cell Proliferation Assay (Promega Corp., Madison, Wis.), as per manufacturer's protocol.

Briefly, 10,000 cells were plated in triplicate in 96 well plates and incubated for 12 hrs in complete media. Compound MK-3 was added as indicated on replacing them to fresh complete media and incubated up to 72 hrs for varied time periods. 20 μl per well of CellTiter 96® AQₒᵤₑᵤₛ One Solution reagent was added. Plates were incubated for 1-4 hrs at 37° C. and humidified 5% CO₂ atmosphere. Absorbance was recorded at 490 nm with 96 well plate readers. Reference wavelength of 630 nm was used to reduce background contributed by non-specific absorbance due to cell debris.

### EXAMPLE 13

### Electrophoresis and Immunoblotting

PC-3 cells (1×10⁶ cells) were incubated with complete medium alone or with 3 μg/ml of Mahanine (MK-3) as indicated. Cells were detached using cell dissociation reagent from Sigma Chemical company St Louis, Mo., USA. The cells were collected by centrifugation at 1500 g for 10 minutes and boiled for 5-7 minutes in SDS buffer (pH 6.8). Aliquots containing 60 μg total cellular protein were separated by 10% SDS-PAGE and transferred to PVDF membrane (MILIPORE, Bedford, Mass., USA). Membrane was blocked with blocking buffer for 1 hr at room temperature and probed with desired primary antibody caspase 9, Bcl-xl, pAkt/1/2/3(ser 473), anti PARP, Cytochrome C (Santa-Cruz Biotechnology Inc., USA). Anti-caspase 3 that recognizes procaspase 3 (32 kDa) and the active cleaved caspase 3 (17 kDa) (BD Biosciences, Mountainview, Calif.) were also used. Akt, cleaved caspase 9 (Cell Signaling Technology, Beverly, Mass.) overnight at 4° C. followed by alkaline phosphatase conjugated secondary antibody and detection.

### EXAMPLE 14

### Immunofluorescence

Cells were cultured on glass cover slips. Both control and treated cells were fixed with PBS containing 4% paraformaldehyde for 2 hrs at 4° C. The cells were permiabilized with 0.1% Triton X-100 in PBS and then incubated with rabbit polyclonal anti-cytochrome c antibody (Santa Cruz, USA, dilution 1:50) for 3 hrs followed by incubation with FITC-conjugated secondary antibody (goat anti-rabbit, Santa Cruz, 1:50) for another 1 hr with rigorous washing in all the above steps with PBS. 1 μg/ml DAPI was also added in each set. The stained cells were observed under fluorescence microscope (Olympus BX51 microscope, Tokyo, Japan) and the images were captured with Cool Snap Pro camera.

### Mahanine Reduces PC-3 Cell Viability by Inducing Apoptosis

Mahanine, purified from HPLC fraction of Murraya koenigii extract, induced death of PC-3 cells. Chemical structure was then determined by using 2D NMR and Mass Spectra and found it to be a mahanine, whose structure was, reported earlier (Tian-shung Wu, 1991, Phytochemistry) (FIG. 1).

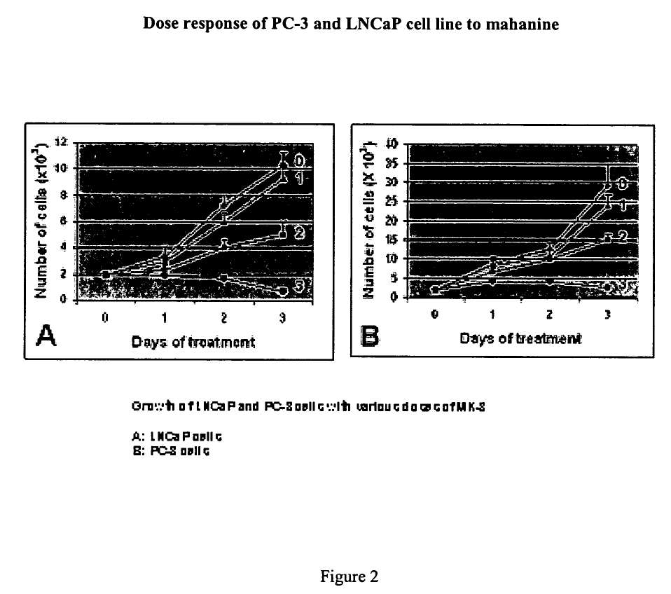

Mahanine killed androgen independent cell line PC-3 and androgen dependent cell line LNCaP cells in a dose and time dependent manner (FIG. 2).

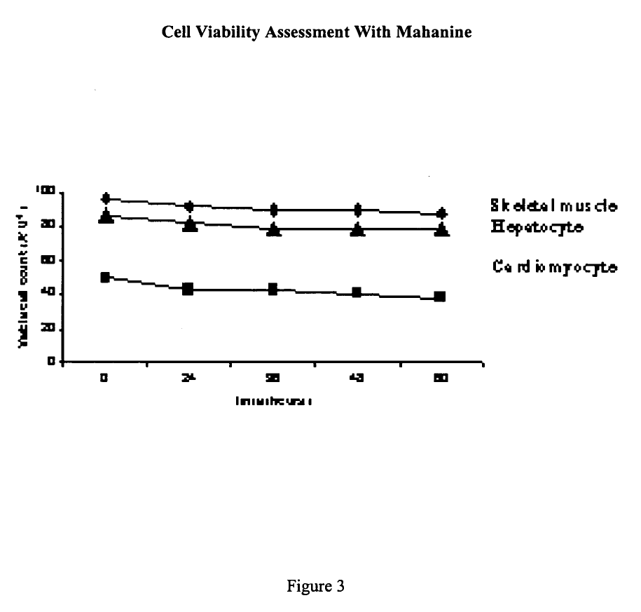

To evaluate the specificity of mahanine, its effect on other cells in primary culture, such as, hepatocytes, cardiomyocyte and skeletal muscle was examined. FIG. 3 shows that mahanine did not kill other cells, thereby suggesting its specificity for prostate cancer cells.

### In Vivo Experiment Confirm In Vitro Results

Experiments for acute toxicity were performed as per the guidelines of WHO.

Experimental subject: Mice; Balb/C Male; female; 25 gms body weight (approx).

16 mice were divided into 2 groups 8 animals were received the compound @2 gm/kg body weight. Equal volume of the vehicle (DMSO) was given to 8 animals, wherein the dose was administered orally once, and then animals were sacrificed and haematological and biochemical parameters checked 15 days after treatment.

TABLE 3(a)
Haematological Tests for Compound Mahanine:

| | RBC | WBC | Hb(gm/dl) |
|---|---|---|---|
| Control | 1029 | 92 | 10.69 |
| Treated | 1032 | 89 | 10.713 |

Biochemical parameters were assessed for the pure molecule mahanine. Since it is a pure molecule there is a need to see the biochemical profile, therefore we concentrated on the biochemical parameters only. The experimental procedure followed was the same as that for the combined extract described above.

TABLE 3(b)
Biochemical tests

| | | | | Creatinine |
|---|---|---|---|---|
| | SGOT(units/ml) | SGPT(units/ml) | Urea(mg/dl) | (mg/dl) |
| Control | 147.5 | 32.0 | 35.4 | 3.82 |
| Treated | 174.0 | 30.7 | 48.6 | 3.72 |

Moreover, there was no indication of any toxicity in mahanine treated mice (Table 3(a) and (b)).

### EXAMPLE 15

### Inhibition of Cell-Survival Pathway in Mahanine Treated PC-3 Cells

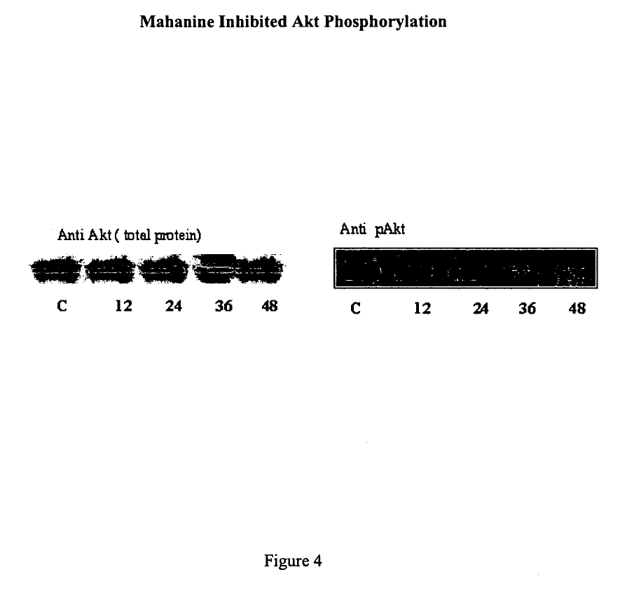

In cell survival pathway, Akt kinase plays an important role by inhibiting apoptotic processes. Akt phosphorylates downstream effector molecules such as pro-apoptotic protein Bad effecting its inactivation. This does not permit its dimerisation with Bcl-XL that result in the inhibition of apoptotic process. Mahanine inhibited phosphorylation of Akt in a dose and time dependent manner. Mahanine showed no effect on the steady state levels of Akt protein but its serine 473 phosphorylation was inhibited at 24 hrs, and by 36 hrs its activation was totally blocked (FIG. 4).

The basal level of phosphorylation at serine 473 is predominant in PC-3 cell line. The inactivation of Akt phosphorylation could be attributed to the inactivation of cell survival pathways resulting subsequent induction of apoptosis in PC-3 cell treated with mahanine.

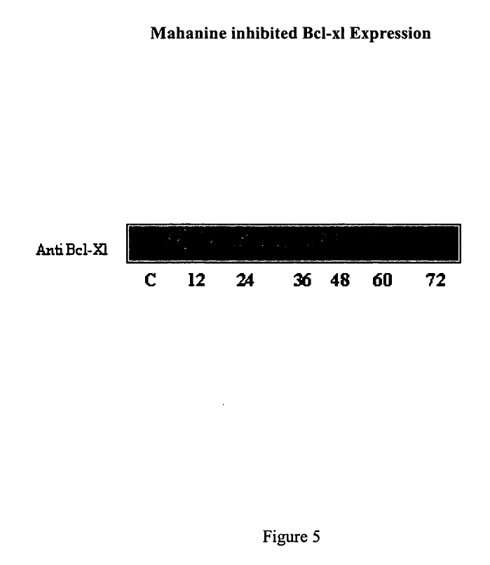

Further studies to determine whether mahanine induced apoptosis of PC-3 cells is affected through Bcl-xl were performed. Bcl-xl is a mitochondrial membrane protein that maintains mitochondrial membrane integrity in survival signal pathway. Mahanine significantly decreased Bcl-xl expression at 36 hrs (FIG. 5).

This indicates initiation of apoptotic pathway as down regulation of Bcl-xl permits disintegration of mitochondrial outer membrane that causes leakage of cytochrome c initiating the caspase cascade. Mahanine caused leakage of cytochrome c from the mitochondria in PC-3 cells.

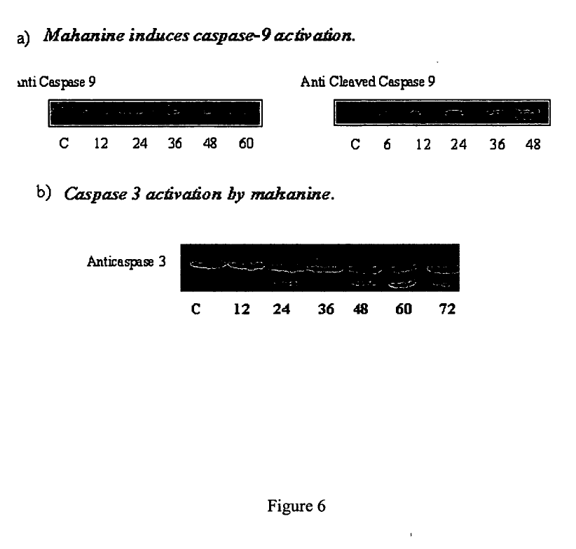

Caspase 9 activation was determined in the same time points with the same dose (3 μg/ml of culture media) of mahanine. Cleaved caspase 9 could be detected as early as 6 hrs, which steadily increased at 12 hrs and 24 hrs, and declined at 36 hrs (FIG. 6a). The activation of caspase 3 considerably increased at 48 hrs, and peak of the activity could be detected at 60 hrs (FIG. 6b).

Mahanine effect on caspase 9 and caspase 3 indicates the activation of death pathway and time of activation of caspase 9 synchronized with caspase 3 which is further downstream in the apoptotic pathway.

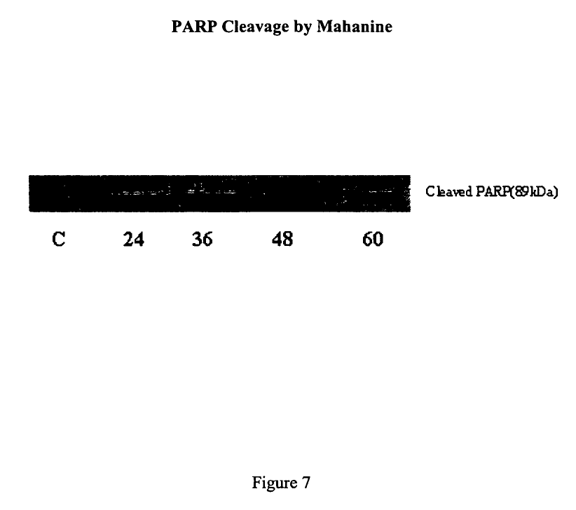

Poly ADP ribosyl Polymerase (PARP) is a DNA repair enzyme responsible for nick detection and repair. It is one of the target proteins of caspase 3. Active caspase 3 causes cleavage of PARP into an 89 kDa fragment in PC-3 cells due to mahanine. The maximum cleavage of PARP was observed at 36 hrs (FIG. 7).

In light of the above results, it may be concluded that mahanine effectively induces apoptosis of both androgen dependent and androgen independent prostate cancer cells.

Furthermore, the present invention includes some advantages. For example, the present invention provides a simplified method of bioactive extraction and a simplified fast and inexpensive process for the preparation of fraction mahanine possessing significantly high biological activities relevant to treatment, relief and remedy of prostate cancer. It also provides a pharmaceutical composition which is highly compatible for human consumption and capable for being used for the treatment, relief, and remedy of prostate cancer.

It is therefore intended that the foregoing detailed description be regarded as illustrative rather than limiting, and that it be understood that it is the following claims, including all equivalents, that are intended to define the spirit and scope of this invention.

1. A pharmaceutical composition for the treatment of prostate cancer in a subject comprising a therapeutically effective amount of a compound mahanine, its derivative, its analogue, or a pharmaceutically acceptable salt of the compound mahanine, or its derivative, or its analogue.

2. The pharmaceutical composition of claim 1, further comprising a pharmaceutically acceptable carrier.

3. The pharmaceutical composition of claim 2, wherein said carrier is selected from the group consisting of proteins, carbohydrates, sugar, magnesium stearate, cellulose, calcium carbonate, and starch-gelatin paste and pharmaceutical acceptable carriers, excipient, diluent, and solvent.

4. The pharmaceutical composition of claim 1, wherein the compound mahanine is obtained from an extract of Murraya Koenigii.

5. The pharmaceutical composition of claim 1, wherein the compound mahanine is synthetically made.

6. The pharmaceutical composition of claim 1, wherein the pharmaceutical composition is in a water-soluble form comprising a unit dose of at least about 0.1-5.0 mg/kg body weight.

7. The pharmaceutical composition of claim 1, wherein the pharmaceutical composition is administered in a water-soluble form.

8. The pharmaceutical composition of claim 1, wherein the pharmaceutical composition is in an oral, intravenous, intramuscular, or subcutaneous formulation.

9. The pharmaceutical composition of claim 8, wherein the oral formulation is selected from the group consisting of a capsule, syrup, concentrate, powder and granules.

10. The pharmaceutical composition of claim 1, wherein the pharmaceutical composition comprises the compound mahanine or its pharmaceutically acceptable salt.

11. A method of treating prostrate cancer in a subject comprising administering to the subject a pharmaceutical composition comprising a therapeutically effective amount of a compound mahanine, its derivative, its analogue, or a pharmaceutically acceptable salt of the compound mahanine, its derivative, or its analogue.

12. The method of claim 11, further comprising administering a pharmaceutically acceptable carrier.

13. The method of claim 12, wherein the carrier is selected from the group consisting of proteins, carbohydrates, sugar, magnesium stearate, cellulose, calcium carbonate, and starch-gelatin paste and pharmaceutical acceptable carriers, excipient, diluent, and solvent.

14. The method of claim 11, wherein the compound mahanine is obtained from an extract of Murraya Koenigii.

15. The method of claim 11, wherein the compound mahanine is synthetically made.

16. The method of claim 11, wherein the step of administering the pharmaceutical composition comprises administering a water-soluble form of the pharmaceutical composition at a unit dose of at least about 0.1-5.0 mg/kg body weight.

17. The method of claim 11, wherein the step of administering further comprises administering the pharmaceutical composition in an oral, intravenous, intramuscular, or subcutaneous formulation.

18. The method of claim 17, wherein the oral formulation is selected from the group consisting of capsule, syrup, concentrate, powder and granules.

19. The method of claim 11, wherein the pharmaceutical composition kills an androgen-independent cell line PC 3 and an androgen-dependent cell line LNCaP, in a dose and time dependent manner.

20. The method of claim 11, wherein the pharmaceutical composition inhibits phosphorylation of Akt in a dose and time dependent manner.

21. A method of isolating compound mahanine from Murraya koenigii, comprising the steps of:
a) extracting fresh leaves of Murraya koenigii with methanol to obtain a methanol extract;
b) concentrating the methanol extract from step (a) to obtain a residue I;
c) suspending the residue I from step (b) in water;
d) extracting the residue I with ethyl acetate and n-butanol to obtain an ethyl acetate layer;
e) concentrating the ethyl acetate layer from step (d) to obtain a residue II;
f) subjecting the residue II from step (e) to chromatography on silica gel using petroleum ether-chloroform (100:00→00:100) as an eluent to obtain fraction 4;
g) subjecting the fraction 4 from step (f) to chromatography on silica gel; and
h) crystallizing the fraction 4 in petrol to obtain the compound mahanine.

22. The method of claim 21, further comprising confirming a structure of the compound mahanine by comparing its physical data and its infrared, NMR and mass spectral data with data from an authentic sample.

23. The method of claim 21, further comprising confirming an anti-carcinogenic activity of the compound mahanine by in vivo experiments.
